(19) United States
(12) Patent Application Publication
(10) Pub. No.: US 2021/0321975 A1
(43) Pub. Date: Oct. 21, 2021

(54) METHODS, SYSTEMS, AND APPARATUSES FOR ANALYZING MUSCULOSKELETAL FUNCTION

(71) Applicant: George Mason University, Fairfax, VA (US)

(72) Inventors: Siddhartha Sikdar, Washginton, DC (US); Parag Chitnis, Burke, VA (US); Biswarup Mukherjee, Fairfax, VA (US); Joseph Majdi, Fairfax, VA (US)

(21) Appl. No.: 17/236,752

(22) Filed: Apr. 21, 2021

Related U.S. Application Data
(60) Provisional application No. 63/013,319, filed on Apr. 21, 2020.

(51) Int. Cl. A61B 8/08; G16H 20/30; G16H 30/40; G06N 20/00

(52) U.S. Cl. CPC A61B 8/08; A61B 8/488; A61B 8/5223; A61N 1/36003; G16H 30/40; G06N 20/00; G16H 20/30

(57) ABSTRACT

A low-power (e.g., battery-operated, etc.) wearable ultrasound system may be used to monitor the musculoskeletal function of a subject and provide information that may be used for electrical stimulation.

### CROSS-REFERENCE TO RELATED PATENT APPLICATIONS

This application claims priority to U.S. Provisional Application No. 63/013,319 filed Apr. 21, 2020, which is hereby incorporated by reference in its entirety.

### GOVERNMENT LICENSE RIGHTS

This invention was made with government support under grant number 1646204 awarded by the National Science Foundation and under grant number W81XWH-16-1-0722 awarded by US Army Medical Research. The government has certain rights in the invention.

### BACKGROUND

Electromyography (EMG) may be used to analyze the function of a user's muscles. EMG signals are noisy and unable to differentiate between different muscle groups. Ultrasound transducers used to analyze the function of a user's muscles are handheld, cumbersome, and subject to operator error. Wearable sensors may detect the heart rate, skin temperature, electrolyte level, and/or the like of a user, but there are no wearable sensors that may selectively analyze the function of a user's individual muscles or deep muscles in real-time. These and other shortcomings are addressed by the present disclosure.

### SUMMARY

Described are methods, systems, and apparatuses for analyzing musculoskeletal function. Low-power ultrasound transducers (sensors) may monitor the musculoskeletal function of a subject. As muscles activate and generate force, they expand radially and/or experience a decline in functionality. Fatigued muscles lose force production and generate force and/or move less. Analysis may be performed on individual muscles, such as deep muscles not readily accessible with conventional methods such as electromyography, to determine instances where a muscular function is declining, a muscle is fatigued, and/or a muscle is recovering. For example, an analysis may be performed based on one or more ultrasonic signals to determine instances where a muscular function is declining, a muscle is fatigued, and/or a muscle is recovering from fatigue, or occurrences of stress and/or injury. Information associated with the function of a subject's muscle may be obtained from one or more wired or wireless low-power ultrasound transducers (sensors) attached to the surface (skin) of the subject. Information associated with the function of a subject's muscle may include quantifications and/or determinations of movement speed, deformation, force generation, fast (twitch) activation, and/or the like. For example, one or more ultrasound transducers may measure the movement speed of muscles that are associated with force generation and/or provide information used to determine changes in muscle activation as a muscle begins to fatigue.

Information associated with the function of a subject's muscle may be used to generate one or more electrical signals. One or more electrical signals may be provided/delivered to the muscle to stimulate and/or promote muscle activity. For example, the information associated with the function of a subject's muscle may be used to provide electrical stimulation to the muscles as needed to promote muscle activity when the muscle is capable of generating force, and stopping electrical stimulation when the muscle is fatigued. Information associated with the function of a subject's muscle may be used to determine one or more signal patterns (e.g., signal strength/amplitude, signal duration, signal frequency, signal timing, etc.) for electrical stimulation using one or more stimulating electrodes that minimize instances of muscle fatigue.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, together with the description, serve to explain the principles of the methods and systems:

FIG. 1 shows an example system for analyzing musculoskeletal function;

FIG. 2 is a diagram of an example ultrasound transducer for analyzing musculoskeletal function;

FIG. 3 shows an example system configuration for analyzing musculoskeletal function;

FIG. 4 shows an example system configuration for analyzing musculoskeletal function;

FIG. 5 is an image for analyzing musculoskeletal function;

FIGS. 6A-6D are diagrams for analyzing musculoskeletal function;

FIG. 7 shows an example method for analyzing musculoskeletal function;

FIG. 8 shows an example method for analyzing musculoskeletal function; and

FIG. 9 shows a block diagram of a computing device for implementing analysis of musculoskeletal function.

### DETAILED DESCRIPTION

Before the present methods and systems are disclosed and described, it is to be understood that the methods and systems are not limited to specific methods, specific components, or particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, solid-state storage, or magnetic storage devices.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Low-power ultrasound transducers (sensors) may monitor the musculoskeletal function of a subject. As muscles activate and generate force, they expand radially and/or experience a decline in functionality. Fatigued muscles lose force production and generate force and/or move less, and/or move in altered patterns. Analysis may be performed on individual muscles, such as deep muscles not readily accessible with conventional methods such as electromyography, to determine instances where a muscular function is declining, a muscle is fatigued, and/or a muscle is recovering. For example, an analysis may be performed based on one or more ultrasonic signals to determine instances where a muscular function is declining, a muscle is fatigued, and/or a muscle is recovering from fatigue, or occurrences of stress and/or injury. Information associated with the function of a subject's muscle may be obtained from one or more wired or wireless low-power ultrasound transducers (sensors) attached to the surface (skin) of the subject. Information associated with the function of a subject's muscle may include quantifications and/or determinations of movement speed, deformation, force generation, fast (twitch) activation, and/or the like. For example, one or more ultrasound transducers may measure the movement speed of muscles that are associated with force generation and/or provide information used to determine changes in muscle activation as a muscle begins to fatigue.

Information associated with the function of a subject's muscle may be used to generate one or more electrical signals. One or more electrical signals may be provided/delivered to the muscle to stimulate and/or promote muscle activity. For example, the information associated with the function of a subject's muscle may be used to provide electrical stimulation to the muscles as needed to promote muscle activity when the muscle is capable of generating force, and stopping electrical stimulation when the muscle is fatigued. Information associated with the function of a subject's muscle may be used to determine one or more signal patterns (e.g., signal strength/amplitude, signal duration, signal frequency, signal timing, etc.) for electrical stimulation using one or more stimulating electrodes that minimize instances of muscle fatigue.

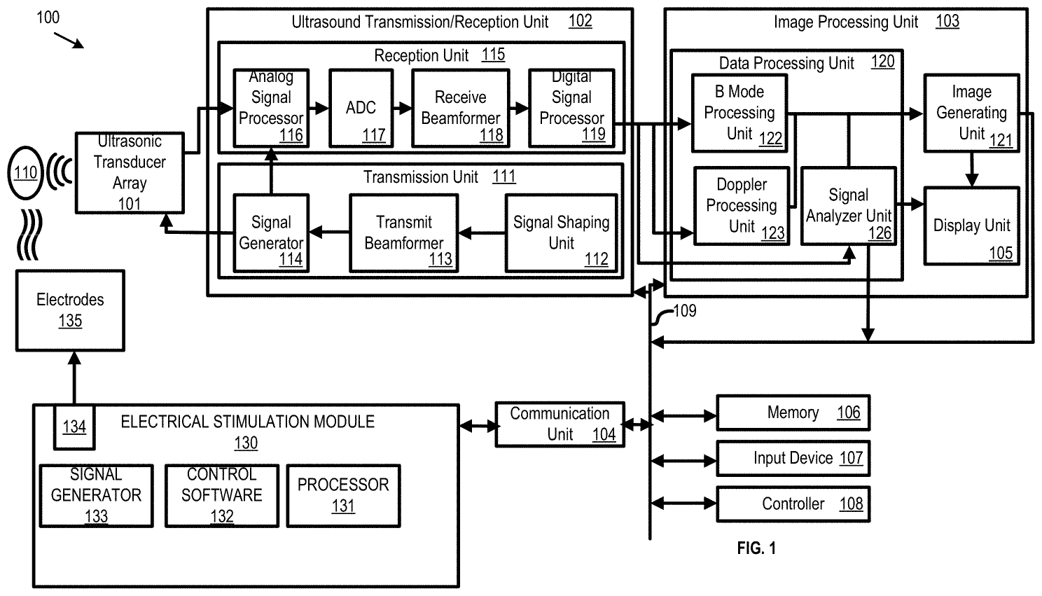

FIG. 1 shows a system 100 for analyzing musculoskeletal function. The system 100 may further include components/configurations not shown in FIG. 1 or may omit some of the components/configurations illustrated in FIG. 1. In some instances, the components/configurations illustrated in FIG. 1 may be substituted by equivalents. In some instances, the system 100 may be a portable battery or power supply (e.g., low power, etc.) operated device, such as a portable type apparatus. The one or more components of the system 100 may be and/or be included with a single device, such as a portable battery or power supply (e.g., low power, etc.) operated device.

The system 100 may include an ultrasound transducer (sensor) array 101, an ultrasound transmission/reception unit (e.g., transceiver) 102, an image processing unit 103, a communication unit 104, a display unit 105, a memory 106, an input device 107, and a controller 108, which may be connected to one another via buses 109. In some instances, system 100 may include multiple ultrasound transducers (not shown), such as the ultrasound transducer 101. The system 100 may include a functional electrical stimulation module 130 including one or more stimulation electrodes 135 and control software 132 running on a processor 131 that communicates with the ultrasound signal and image processing unit 103 through a communication unit 104.

The combination of the ultrasound and electrical stimulation modules can be used to determine the twitch response of muscle, and determine the state of the muscle, whether it is fatigued, or able to generate force. In some implementations, the image generation unit 121 display unit 105 and the input device 107 may be omitted, and the signal analyzer unit 126 may directly communicate through the memory 106 with the controller 108 and communication unit 104 to control the electrical stimulation unit.

The ultrasound transducer 101 may transmit ultrasound waves to an object 110. The object 110 may include musculoskeletal tissue, a muscle group (e.g., quadriceps or other muscle groups in the body, one or more muscles overlying each other, etc.), and/or the like. The ultrasound transducer 101 may transmit ultrasound waves to an object 110 in response to a driving signal applied by the ultrasound transmission/reception unit 111. The ultrasound transducer 101 may receive backscattered signals reflected by the object 110. The ultrasound transducer 101 may include a plurality of transducers that oscillate in response to electric signals and generate acoustic energy, such as ultrasound waves. For example, the ultrasound transducer 101 may be a miniature transducer array that includes poly(vinylidene fluoride-tetrafluoroethylene) (P(VDF-TrFE)) film.

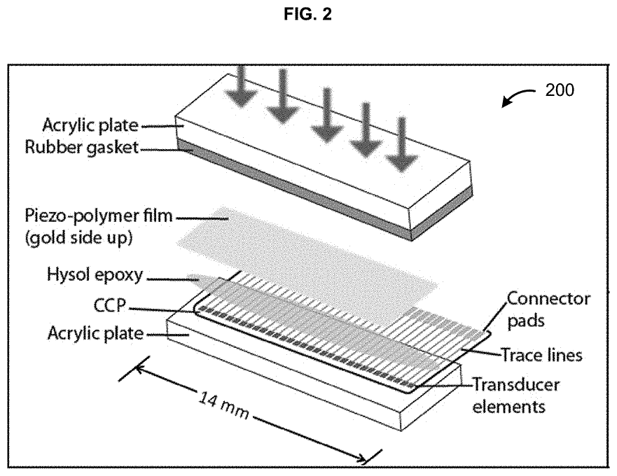

FIG. 2 is a diagram of an example ultrasound transducer 200 (e.g., the ultrasound transducer 101, etc.) A piezo-polymer film may be attached to electrode tracks etched into copper-clad-polyimide (CCP). A backing material may be used to dampen reflections and increase the bandwidth and/or spatial resolution of the transducer 200. Transducer elements may be connected to and or in communication with the ultrasound transducer 101 and/or any other component of the system 100.

Returning to FIG. 1, the ultrasound transmission/reception unit 102 may be in communication with the image processing unit 103. In some instances, the ultrasound transmission/reception unit 102 may be in communication with the image processing unit 103 via a wireless connection and/or communication medium. In some instances, the ultrasound transmission/reception unit 102 may be in communication with the image processing unit 103 via a wired connection and/or communication medium.

A transmission unit 111 may provide/supply a driving signal to the ultrasound transducer 101. The transmission unit 111 may include a signal shaping unit 112, a transmit beamformer 113, and a signal generator 114. The signal shaping unit 112 may generate pulses used to form transmission ultrasound waves based on a predetermined pulse repetition frequency (PRF), may generate continuous wave signals, and/or may generate frequency-modulated continuous-wave signals with appropriate parameters. For example, the ultrasound transmission/reception unit 102 may use frequency-modulated continuous-wave signals to perform time-delay spectroscopy. The transmit beamformer 113 may delay pulses generated by the signal shaping unit 112 by periods/times/durations necessary for determining transmission directionality and focusing, or might perform time-frequency domain beamforming of frequency-modulated signals. The ultrasound transducer 101 may include a plurality of piezoelectric vibrators. Delayed pulses or frequency-encoded signals may correspond to and/or be associated with the plurality of piezoelectric vibrators. The signal generator 114 may apply driving signals and/or driving pulses to the ultrasound transducer 101 based on timing corresponding to each of the pulses which have been delayed, or frequency-encoded. In some implementations, the transducer array 101 may consist of only one element, and the transmit beamformer 113 might be omitted.

A reception unit 115 may generate ultrasound data by processing backscattered signals received from the ultrasound transducer 101. The reception unit 115 may include an analog signal processor 116, an analog-to-digital converter (ADC) 117, a receive beamformer 118, and a digital signal processor 119. The analog signal processor 116 may amplify backscattered signals in each channel. Optionally, the analog signal processor 116 may perform demodulation of the received signal to baseband using the reference transmit signal from the signal generator 114, or using an. alternative demodulation method. The analog signal processor 116 may also perform filtering of the signal. The ADC 117 may perform analog-to-digital conversion with respect to the processed signals produced by the analog signal processor 116. The receive beamformer 118 may delay digital signals output by the ADC 117 by delay periods/times/durations necessary for determining reception directionality and focusing, and sum the delayed signals to produce a beamformed signal. Alternatively, frequency-domain beamforming may be performed for frequency-modulated and frequency-encoded signals. In some implementations, where the transducer 101 consists of a single element, the beamforming unit 118 may be omitted. The digital signal processor 119 processes the digital signals further, for example, to perform a Fast Fourier transform for frequency-modulated received signals with a single element transducer for time-delay spectroscopy imaging. In some implementations, the digital signal processor 119 may be omitted if further processing after beamforming is not necessary.

The image processing unit 103 generates one or more ultrasound images by processing ultrasound data generated by the ultrasound transmission/reception unit 102. The image processing unit 103 may include a data processing unit 120 and an image generating unit 121. The image processing unit 103 may process the ultrasound data. For example, the image processing unit 103 may process the ultrasound data via any method, such as image reconstruction and/or filtering. In some instances, the ultrasound image may be a grayscale ultrasound image obtained by scanning an object in an amplitude (A) mode, a brightness (B) mode, and a motion (M) mode. In some instances, the ultrasound image may be generated by a Doppler signal showing the movement of an object via a Doppler effect. The Doppler image may be of a muscle or surrounding tissue, such as an image of muscle and tissue before and after biphasic twitch stimuli. The image processing unit 103 may also analyze the image sequences to automatically calculate metrics of muscle function.

The data processing unit 120 can comprise a B mode processing unit 122 and/or a Doppler processing unit 123. The B mode processing unit 122 extracts B mode components from ultrasound data and processes the B mode components. The image generating unit 121 may generate an ultrasound image indicating signal intensities as brightness based on the extracted B mode components. The Doppler processing unit 123 may extract Doppler components from the ultrasound data. The image generating unit 121 may be based on the extracted Doppler components, generate a Doppler image indicating a movement of an object as colors or waveforms.

The data processing unit 120 may include a signal analyzer unit 126. The signal analyzer unit 126 may receive ultrasound data from the reception unit 115. The signal analyzer unit 126 may analyze the ultrasound data to compare ultrasound reflections from a muscle (pre-stimulus and post-stimulus), different muscles, different muscle groups, and/or the like to measure muscle contraction and assess muscle fatigue. For example, the ultrasound data may be used to analyze muscle fiber velocity, such as a peak velocity, a time to zero velocity, a twitch duration, peak displacement, and/or the like. In some instances, ultrasound data from the reception unit 115 may be provided to a trained machine learning model and/or artificial intelligence algorithm configured to determine patterns associated with muscle activity, such as twitches in muscle tissue.

Twitches in muscle tissue may indicate the onset of muscle fatigue and/or fatigue recovery. The data processing unit 120 may include a trained machine learning model. The machine learning model may predict muscle fatigue or any other condition based on image data and/or any other parameters/information associated with muscle tissue. The machine learning model may be used to determine muscle fatigue and/or muscle force.

In some instances, the signal analyzer unit 126 may estimate an instantaneous intensity of backscattered ultrasound signals from muscle tissue. The analog signal processor 116 conditions the analog backscattered signal for digitization by the ADC 117 through the operations of amplification and filtering. The received signal may be demodulated and converted to quadrature components either in the analog signal processor 116 or digital signal processor 119. The received analog signal may be demodulated by mixing with the transmitted signal and filtering to preserve phase information or using another demodulation method, such as a diode demodulator, when phase information is not desired. The received digital signal may be converted to analytic form (complex-valued), for example, via a Hilbert transform or quadrature filtering operation. If an analytical signal is utilized, the magnitude of the complex-valued received signal may be defined as the instantaneous backscattered intensity and may be summed over spatially adjoining samples in depth and/or lateral directions to comprise one image voxel in the B-mode processing unit 122. If a frequency-modulated signal is utilized, the magnitude of the demodulated and filtered signal may be processed by a Fast Fourier Transform (or alternative spectral analysis method), and the spectral power at a given frequency may be defined as the instantaneous backscattered intensity at a corresponding depth. The instantaneous backscattered intensity may be summed over spatially adjoining samples in depth and/or lateral directions to comprise one image voxel in the B-mode processing unit 122. The instantaneous backscattered intensity may be calculated for every voxel in the field of regard of the ultrasound transmission. The time course of the instantaneous backscattered intensity in each voxel may exhibit cyclic oscillations with a period equal to that of muscle stimuli (e.g., volitional stimulus, electrical stimulus, induced electrical stimulus, etc.). The backscattered signals may be processed in the Doppler processing unit 123 to determine the instantaneous muscle speed, strain, or strain rate using phase-domain or frequency-domain Doppler processing methods. The time courses for instantaneous backscattered intensity measures and/or the instantaneous muscle speed, strain, or strain rate may be utilized by the signal analysis unit 126 to determine a muscle activation signal. The muscle activation signal from multiple cycles may be time-synchronized and averaged together. The duration of a stimulus cycle can also be derived from the intrinsic period of the signals derived from the muscle activation signal. The time course, and intrinsic features thereof, of the muscle activation signal across the stimulus cycle, are compared for each voxel against: 1) previously-stored known variations in normal muscle tissue, 2) previously-stored patterns or time courses of fatigue and/or stimulated muscle tissue, 3) previously-stored historical patterns or time courses from the same subject, if available, for monitoring treatment (e.g., fatigue recovery, etc.). Any number of classification algorithms (including but not limited to Bayesian, neural network, support vector machines, k-nearest neighbor, and binary decision) can be used to determine whether the observed muscle tissue region exhibits, when stimulated, (a) normal activation, (b) abnormal activation characteristic of muscle tissue (e.g., caused/affected by fatigue/injury, etc.), (c) normal twitch response or (d) abnormal twitch response (e.g., caused/affected by fatigue/injury, etc.).

The image generating unit 121 may, based on the ultrasound information, generate images, such as two-dimensional (2D) images and/or three-dimensional (3D) images. For example, the image generating unit 121 may generate a three-dimensional (3D) ultrasound image via volume-rendering with respect to volume data. The display unit 105 may display various pieces of additional information in an ultrasound image by using text and graphics. For example, the display unit 105 may display muscle identification data/information and/or the like. In some implementations, the image generation unit 121 and the display unit 105 may be omitted.

Ultrasound images may be stored in the memory 106. The display unit 105 displays the generated ultrasound image. The display unit 105 may display not only an ultrasound image, but also various pieces of information processed by the system 100 on a screen image via a graphical user interface (GUI). The display unit 105 may display one or more results of the signal analyzer unit 126 or image generating unit 121. In some instances, the display unit 105 may display one or more of a composite spatial map of muscle tissue potentiation, twitch, movement, and/or the like. In some instances, the display unit 105 may display a parametric spatial map indicating whether different muscles and/or muscle groups exhibit potentiation and tissue properties that are (a) normal, (b) characteristic of fatigue, (c) characteristic of muscle recovery, or (d) indeterminate.

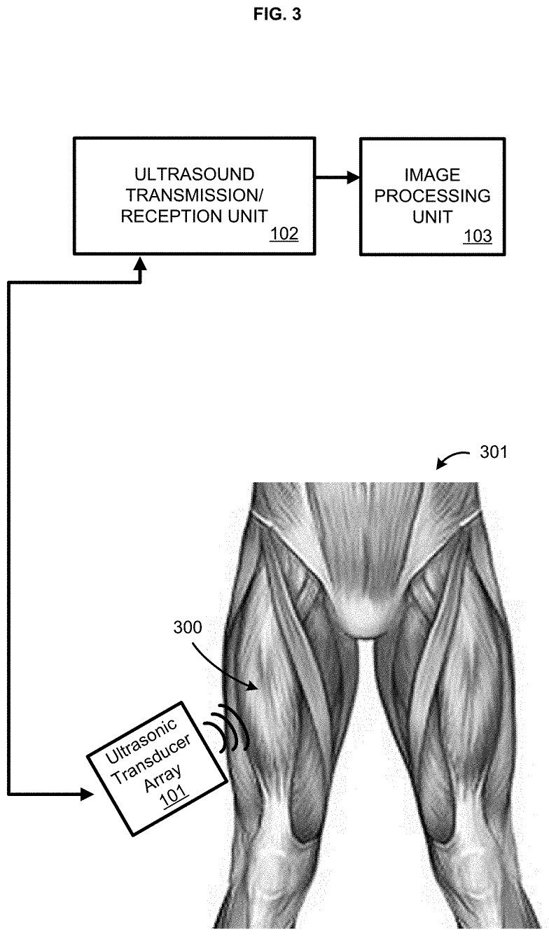

FIG. 3 illustrates an example configuration for the system 100. The object 110 can include a muscle group 300. The muscle group 300 may include the rectus femoris (RF) and vastus intermedius (VI) muscles of a subject 301. The ultrasound transducer 101 may be placed on the surface (skin) of the subject 301. The ultrasound transducer 101 may be coupled to the ultrasound transmission/reception unit 102 and may operate as disclosed herein.

In some instances, the information displayed on the display unit 105 may be used to determine and/or monitor muscle fatigue and muscle recovery of a subject. Information associated with muscle fatigue and muscle recovery may be used to determine and/or identify muscles that may benefit from electrical stimulation.

In some instances, the system 100 may include an electrical stimulation (ES) module 130. The ES module 130 may be a battery and/or power supply operated device. The communication unit 104 may be in either wired or wireless communication with the ES module 130. The communication unit 104 may exchange data/information with the ES module 130, such as determined (monitored) instances of muscle fatigue and/or recovery.

The ES module 130 may comprise a processor 131 in communication with a signal generator 133. The ES module 130 may comprise control software 132 configured for controlling the performance of the processor 131 and/or the signal generator 133. The performance and/or operation of the processor 131 and/or the signal generator 133 may be based on data/information received from the communication unit 104.

The signal generator 133 may generate one or more electric signals in the shape of waveforms or trains of pulses based on determined (monitored) instances of muscle activity (e.g., muscle fatigue, muscle recovery, etc.). The signal generator 133 may generate any electrical signal that may be used to stimulate the object 110 (e.g., stimulate a muscle).

One or more outputs 134 of the ES module 130 may be coupled to one or more conductive leads that are attached at one end thereof to the signal generator 133. The opposite ends of the conductive leads may be connected to one or more electrodes 135 that are activated by the electric signals. The conductive leads may comprise standard isolated conductors with a flexible metal shield and may be grounded to prevent the spread of the electrical field generated by the conductive leads. The one or more outputs 134 may be operated sequentially. Output parameters of the signal generator 133 may comprise, for example, an intensity/amplitude, frequency, timing, and/or the like of the electric signal and/or any other parameter associated with electrical stimulation. The output parameters may be set and/or determined by the control software 132 in conjunction with the processor 131. After determining a desired electrical signal, the control software 132 may cause the processor 131 to send a control signal to the signal generator 133 that causes the signal generator 133 to output the desired electrical signal to the electrodes 135. In some instances, information associated with the function of a subject's muscle, determined from signal analyzer unit 126, may be used to determine one or more signal patterns (e.g., signal strength/amplitude, signal duration, signal timing, signal frequency, etc.) to be generated by the ES module 130 and output by the electrodes 135 that minimize instances of muscle fatigue.

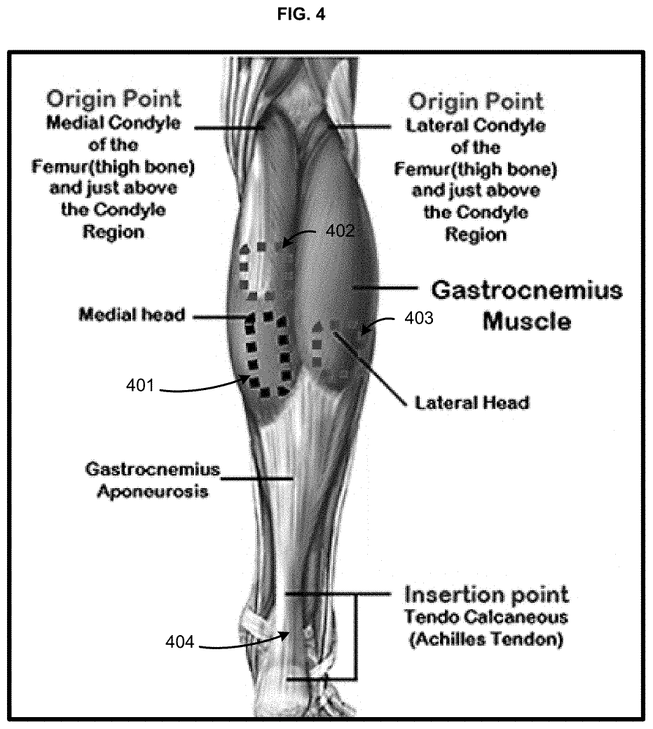

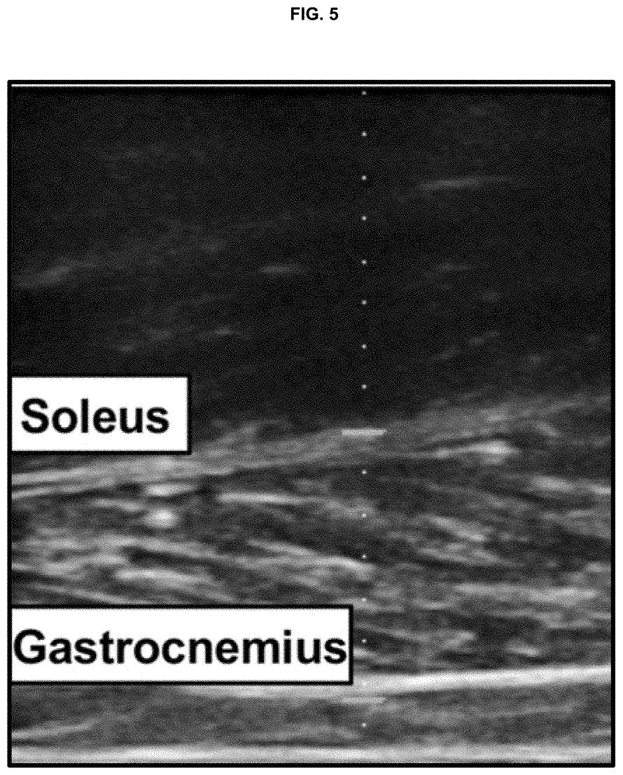

FIG. 4 illustrates an example configuration for the system 100. In some instances, the ultrasound transmission/reception unit 102 may be used to monitor muscle activity of a subject and the system 100 may determine a condition of the muscle (e.g., muscle fatigue, muscle recovery, etc.) during instances of stimulation, such as functional or neuromuscular electrical stimulation. The ultrasound transducer array 101 may be placed on the surface (skin) of a subject that covers one or more muscles. For example, the ultrasound transducer array 101 may be placed at the location 401. The location 401 may be an area over the medial head of the subject's gastrocnemius muscle. The signal analyzer unit 126 may generate signals that are used to monitor activity of the subject's gastrocnemius muscle, and any other deep layer muscle, such as the subject's soleus muscle. FIG. 5 is an image of the subject's gastrocnemius muscle and soleus muscle produced by B-mode imaging.

Returning to FIG. 4, the electrodes 135 may be placed at different locations on the subject's muscles. For example, the electrodes 135 may be placed on the skin over the medial and lateral heads of the subject's gastrocnemius muscle which are identified by the location 402 and 403, respectively. The signal analyzer unit 126 may generate signals that are used to monitor activity of the subject's gastrocnemius muscle and soleus muscle during the application twitch stimuli (electrical signal), such a biphasic twitch stimuli (electrical signal) generated by the ES module 130 before and/or after sustained muscle contraction. The signal analyzer unit 126 may generate signals that are used to monitor activity of the subject's gastrocnemius muscle and soleus muscle while the muscles are stimulated to a sustained muscle contraction at a percentage (e.g., twenty percent, etc.) of maximum force for a duration, such as 60 seconds. The signal analyzer unit 126 may generate signals that are used to monitor activity of the subject's gastrocnemius muscle and soleus muscle before, during, and after the stimulation duration and the muscles are potentially fatigued. The signal analyzer unit 126 may determine spectral content (velocity), twitch duration, and/or the like associated with the subject's muscles (gastrocnemius muscle and soleus muscle). The spectral content (velocity), twitch duration, and/or the like associated with the subject's muscles may be used to determine instances of potentiation, fatigue induced by a stimulus (or occurring naturally), as well as recovery from fatigue.

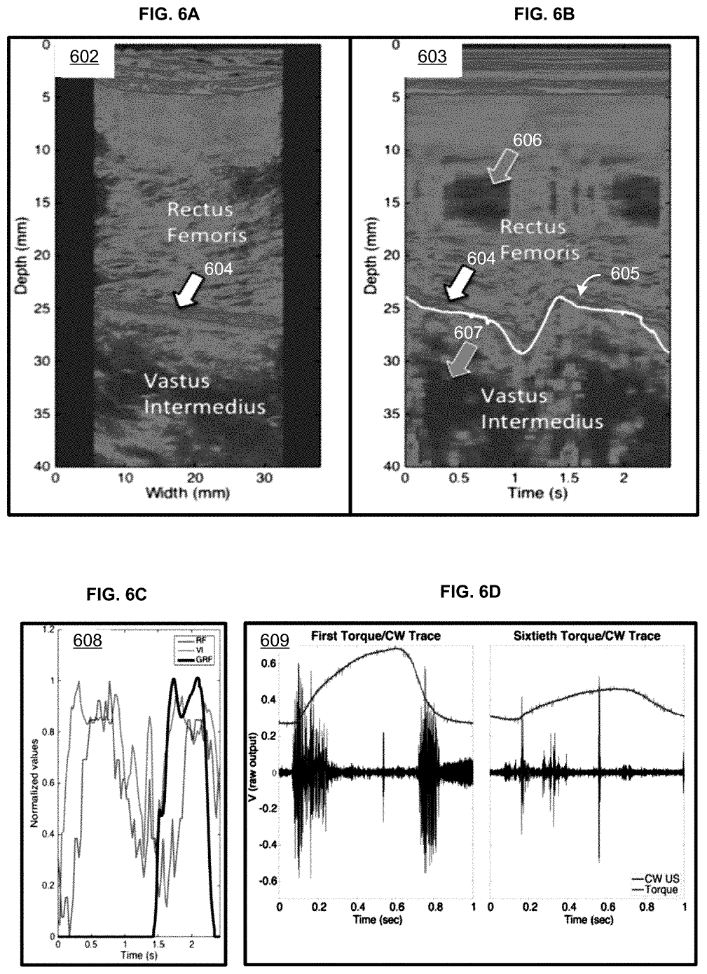

FIGS. 6A and 6B illustrate images 602 and 603 of muscle activity captured by B-mode and M-mode imaging, respectively. The images 602 and 603 depict activity of the rectus femoris (RF) and vastus intermedius (VI) muscles of a subject where force plates were used to measure ground reaction forces (GRF). The image 602 is a B-mode ultrasound image showing the RF and VI muscles. The arrow 604 indicates the aponeurosis of the RF. The image 603 is an M-mode ultrasound image along the center scan line of image 602 over time. The thickness of the RF muscle changes during the gait cycle, and can be seen by following the trace of the aponeurosis (indicated by the arrow 604) over time. A depiction of a right knee angle is overlaid over the image 603 (trace 605). The trace 605 indicates ideal agreement with the change in muscle thickness over time. The echogenicity in the muscle belly of the RF (indicated by the arrow 606) and VI (indicated by the arrow 607) decrease at different times during the gait cycle indicating contraction.

FIG. 6C depicts chart 608. The chart 608 illustrates traces of the normalized echogenicity (inverted for convenient interpretation) of the RF and VI during the gait cycle. The chart 608 also illustrates a GRF trace. The chart 608 illustrates that the RF and VI are active at different points during the gait cycle. FIG. 6D depicts chart 609. The chart 609 provides an ultrasound-based assessment of fatigue during plantar flexion induced by electrical stimulation. The chart 609 illustrates that after sixty seconds of electrical stimulation, the soleus and gastrocnemius calf muscles have fatigued (right panel), generating less torque, in comparison to the first stimulation sequence (left panel). The results depicted by the chart 609 corresponds to a decrease in continuous wave Doppler ultrasound signal amplitude, frequency, timing, and duration at both the onset and end of force generation. In some instances, the Doppler signal can be played as an audio signal to provide real-time “bio-feedback” to a user.

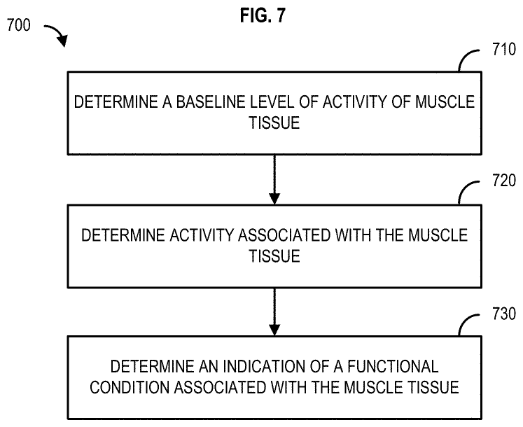

FIG. 7 is a flowchart of an example method 700 for analyzing musculoskeletal function. At 710, a baseline level of activity of muscle tissue associated with a subject may be determined. The baseline level of activity of the muscle tissue may be determined based on information associated with one or more ultrasound signals and/or ultrasound images. For example, a wearable low-power (e.g., battery-powered, etc.) miniature (e.g., dimensions ranging from 0 mm-50 mm, etc.) ultrasound system (e.g., the ultrasound system 100, etc.) may be constructed using ultrasound transducer or transducers 101 composed of a flexible piezoelectric co-polymer and polyamide substrate with conductive micro-patterns. The ultrasound transducer may be attached to the skin of the subject over the muscle tissue. An ultrasound control system (e.g., the system 100, etc.) may cause ultrasonic signals to be transmitted by the ultrasound transducer targeting the muscle tissue. Continuous-wave Doppler imaging may be used to generate one or more ultrasound signals. Other imaging methods, such as pulse-wave imaging or frequency-modulated continuous-wave imaging, or stepped frequency-modulated imaging may also be used to generate one or more ultrasound signals. The one or more ultrasound signals (and/or images) depict information associated with the muscle tissue and/or information associated with the muscle tissue may be determined from the one or more ultrasound signals and used to determine and/or represent the baseline level of activity.

At 720, activity associated with the muscle tissue may be determined. The activity associated with the muscle tissue may be determined based on information associated with another one or more ultrasound signals from imaging of the muscle and/or surrounding tissue. The activity associated with the muscle tissue may include velocity, strain, strain rate, twitch amplitude, twitch duration, or peak displacement. In some instances, the velocity, strain, strain rate, twitch amplitude, twitch duration, or peak displacement may be induced by one or more electrical signals (e.g., electrical stimulation, etc.). The one or more electrical signals may be associated with an amplitude, frequency, timing, and/or signal duration determined based on the baseline level of activity or any other method.

At 730, an indication of a functional condition associated with the muscle tissue may be determined. The indication of the functional condition may be determined based on comparing the baseline level of activity to the activity associated with the muscle tissue. The functional condition of the muscle tissue may be instances of fatigue and/or recovery. In some instances, based on the information associated with ultrasound imaging signals, one or more of the amplitude, frequency, timing, and/or signal durations associated with the one or more electrical stimulation signals may be adjusted/modified, such as to promote activity of the muscle tissue during electrical stimulation.

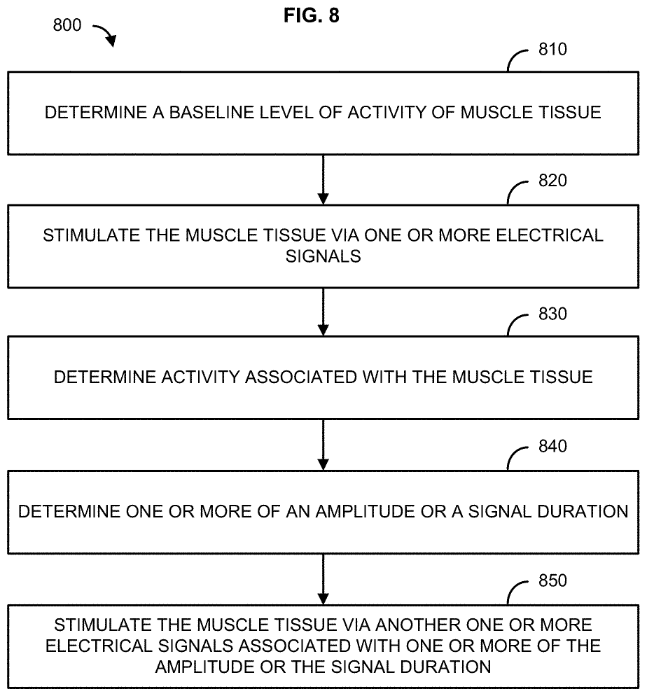

FIG. 8 is a flowchart of an example method 800 for analyzing musculoskeletal function. At 810, a baseline level of activity of muscle tissue associated with a subject may be determined. The baseline level of activity of the muscle tissue may be determined based on information associated with one or more ultrasound images and/or signals. For example, a wearable low-power (e.g., battery-powered, etc.) miniature (e.g., dimensions ranging from 0 mm-50 mm, etc.) ultrasound system (e.g., the ultrasound system 100, etc.) may be constructed using ultrasound transducer or transducers 101 composed of a flexible piezoelectric co-polymer and polyamide substrate with conductive micro-patterns. The ultrasound transducer may be attached to the skin of the subject over the muscle tissue. An ultrasound control system (e.g., the system 100, etc.) may cause ultrasonic signals to be transmitted by the ultrasound transducer targeting the muscle tissue. Continuous-wave Doppler imaging may be used to generate the one or more ultrasound signals (and/or images). The one or more ultrasound signals (and/or images) include information associated with the muscle tissue and/or information associated with the muscle tissue may be determined from the one or more ultrasound signals (and/or images) and used to determine and/or represent the baseline level of activity.

At 820, the muscle tissue may be stimulated. For example, electrical stimulation techniques/methods may be used. One or more ultrasound signals may be associated with one or more parameters, such as a velocity, a strain quantity, a strain rate, a twitch amplitude, a twitch duration, and a peak displacement, determined based on the baseline level of activity or any other method. The one or more electrical stimulation signals may be provided to (applied) to the muscle tissue via one or more electrodes (e.g., the electrode 135, etc.), such as one or more electrodes placed above the skin overlaying or implanted within the muscle tissue. The one or more electrical stimulation signals may be used to activate the muscle tissue based on a signal amplitude or signal duration.

At 830, activity associated with the muscle tissue may be determined. The activity associated with the muscle tissue may be determined based on information associated with another one or more ultrasound signals (and/or images) from imaging of the muscle and/or surrounding tissue. The activity associated with the muscle tissue may include potentiation, velocity, strain, strain rate, twitch amplitude, twitch duration, or peak displacement. The potentiation, velocity, strain, strain rate, twitch amplitude, twitch duration, or peak displacement may be induced by the one or more electrical stimulation signals.

At 840, an amplitude, frequency, timing, and/or signal duration may be determined for another one or more ultrasound signals. The amplitude, frequency, timing, and/or signal duration may be determined based on the activity associated with the muscle tissue. For example, based on the potentiation, velocity, strain, strain rate, twitch amplitude, twitch duration, or peak displacement induced by the one or more electrical stimulation signals, a determination/indication of a functional condition associated with the muscle tissue may be determined. The indication of the functional condition may be determined based on comparing the baseline level of activity to the activity associated with the muscle tissue. The functional condition of the muscle tissue may be instances of fatigue and/or recovery. One or more of the amplitude, frequency, timing, and/or the signal duration associated with the one or more electrical stimulation signals may be adjusted/modified based on one or more received ultrasound signals (and/or images), such as one or more parameters associated with the received ultrasound signals (and/or images). The one more parameters may include a velocity, a strain quantity, a strain rate, a twitch amplitude, a twitch duration, and a peak displacement associated with the muscle tissue.

At 850, the muscle tissue may be activated based on one or more electrical stimulation signals. The adjusted/modified electrical stimulation signals, based on the one more parameters, may be used to promote activity of the muscle tissue during electrical stimulation.

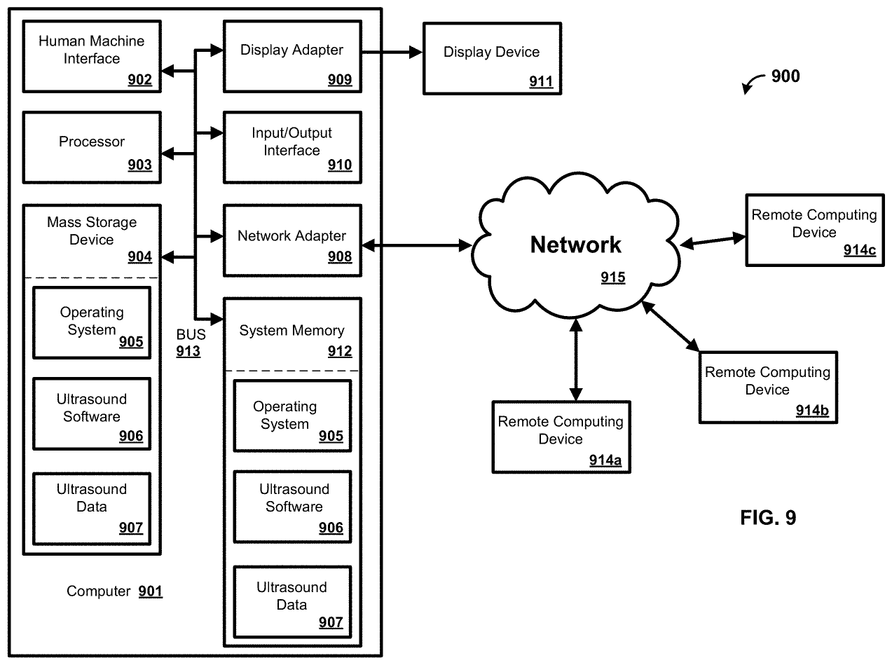

The methods and systems can be implemented on a computer 901 as illustrated in FIG. 9 and described below. In some instances, any device, system, and/or component described herein can be a computer 901 as illustrated in FIG. 9. Similarly, the methods and systems disclosed can utilize one or more computers to perform one or more functions in one or more locations. FIG. 9 is a block diagram illustrating an exemplary operating environment 900 for performing the disclosed methods. This exemplary operating environment 900 is only an example of an operating environment and is not intended to suggest any limitation as to the scope of use or functionality of operating environment architecture. Neither should the operating environment 900 be interpreted as having any dependency or requirement relating to any one or combination of components illustrated in the exemplary operating environment 900.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, multiprocessor systems, processors in a smartphone or similar handheld device, as well as embedded processors. Additional examples comprise set-top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computer 901. The computer 901 can comprise one or more components, such as one or more processors 903, a system memory 912, and a bus 913 that couples various components of the computer 901 including the one or more processors 903 to the system memory 912. In the case of multiple processors 903, the system can utilize parallel computing.

The bus 913 can comprise one or more of several possible types of bus structures, such as a memory bus, memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 913, and all buses specified in this description can also be implemented over a wired or wireless network connection and one or more of the components of the computer 901, such as the one or more processors 903, a mass storage device 904, an operating system 905, ultrasound software 906, ultrasound data 907, a network adapter 908, system memory 912, an Input/Output Interface 910, a display adapter 909, a display device 911, and a human-machine interface 902, can be contained within one or more remote computing devices 914a,b,c at physically separate locations, connected through buses of this form, in effect implementing a fully distributed system.

The computer 901 typically comprises a variety of computer-readable media. Exemplary readable media can be any available media that is accessible by the computer 901 and comprises, for example, and not meant to be limiting, both volatile and non-volatile media, removable and non-removable media. The system memory 912 can comprise computer-readable media in the form of volatile memory, such as random access memory (RAM), and/or non-volatile memory, such as read-only memory (ROM). The system memory 912 typically can comprise data such as ultrasound data 907 and/or program modules such as operating system 905 and ultrasound software 906 that are accessible to and/or are operated on by the one or more processors 903.

In another aspect, the computer 901 can also comprise other removable/non-removable, volatile/non-volatile computer storage media. The mass storage device 904 can provide non-volatile storage of computer code, computer-readable instructions, data structures, program modules, and other data for the computer 901. For example, a mass storage device 904 can be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Optionally, any number of program modules can be stored on the mass storage device 904, including by way of example, an operating system 905 and ultrasound software 906. One or more of the operating system 905 and ultrasound software 906 (or some combination thereof) can comprise elements of the programming and the ultrasound software 906. Ultrasound data 907 can also be stored on the mass storage device 904. Parameters derived from the ultrasound data 907 can be stored in any of one or more databases known in the art. Examples of such databases comprise, DB2®, Microsoft® Access, Microsoft® SQL Server, Oracle®, MySQL, PostgreSQL, SQLite, and the like. The databases can be centralized or distributed across multiple locations within the network or local to the device itself. 915.

In another aspect, the user can enter commands and information into the computer 901 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices can be connected to the one or more processors 903 via a human-machine interface 902 that is coupled to the bus 913, but can be connected by other interface and bus structures, such as a parallel port, game port, an IEEE 1394 Port (also known as a Firewire port), a serial port, network adapter 908, and/or a universal serial bus (USB).

In yet another aspect, a display device 911 can also be connected to the bus 913 via an interface, such as a display adapter 909. It is contemplated that the computer 901 can have more than one display adapter 909 and the computer 901 can have more than one display device 911. For example, a display device 911 can be a monitor, an LCD (Liquid Crystal Display), light emitting diode (LED) display, television, smart lens, smart glass, and/or a projector. In addition to the display device 911, other output peripheral devices can comprise components such as speakers (not shown) and a printer (not shown), which can be connected to the computer 901 via Input/Output Interface 910. Any step and/or result of the methods can be output in any form to an output device. Such output can be any form of visual representation, including, but not limited to, textual, graphical, animation, audio, tactile, and the like. The display 911 and computer 901 can be part of one device, or separate devices.

The computer 901 can operate in a networked environment using logical connections to one or more remote computing devices 914a,b,c. By way of example, a remote computing device 914a,b,c can be a personal computer, computing station (e.g., workstation), portable computer (e.g., laptop, mobile phone, tablet device), smart device (e.g., smartphone, smartwatch, activity tracker, smart apparel, smart accessory), security and/or monitoring device, a server, a router, a network computer, a peer device, edge device or other common network nodes, and so on. Logical connections between the computer 901 and a remote computing device 914a,b,c can be made via a network 915, such as a local area network (LAN) and/or a general wide area network (WAN). Such network connections can be through a network adapter 908. A network adapter 908 can be implemented in both wired and wireless environments. Such networking environments are conventional and commonplace in dwellings, offices, enterprise-wide computer networks, intranets, and the Internet.

For purposes of illustration, application programs and other executable program components such as the operating system 905 are illustrated herein as discrete blocks, although it is recognized that such programs and components can reside at various times in different storage components of the computing device 901, and are executed by the one or more processors 903 of the computer 901. An implementation of ultrasound software 906 can be stored on or transmitted across some form of computer-readable media. Any of the disclosed methods can be performed by computer readable instructions embodied on computer-readable media. Computer-readable media can be any available media that can be accessed by a computer. By way of example and not meant to be limiting, computer-readable media can comprise “computer storage media” and “communications media.” “Computer storage media” can comprise volatile and non-volatile, removable and non-removable media implemented in any methods or technology for storage of information such as computer-readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In view of the described apparatuses, systems, and methods and variations thereof, herein below are described certain more particularly described embodiments of the invention. These particularly recited embodiments should not however be interpreted to have any limiting effect on any different claims containing different or more general teachings described herein, or that the “particular” embodiments are somehow limited in some way other than the inherent meanings of the language literally used therein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
determining, based on information associated with one or more of ultrasound images and ultrasound signals, a baseline level of activity of muscle tissue associated with a subject;
determining, based on information associated with another one or more of ultrasound images and ultrasound signals, activity associated with the muscle tissue; and
determining, based on the baseline level of activity and the activity associated with the muscle tissue, an indication of a functional condition associated with the muscle tissue.

2. The method of claim 1, wherein the indication of the functional condition comprises one or more of an indication of fatigue associated with the muscle tissue or recovery associated with the muscle tissue.

3. The method of claim 1 further comprising causing, based on one or more electrical stimulation signals, the activity associated with the muscle tissue.

4. The method of claim 3 further comprising determining, based on the information associated with the one or more of ultrasound images and ultrasound signals or the information associated with the another one or more of ultrasound images and ultrasound signals, one or more of an amplitude, a frequency, a timing, a metric output by a machine learning model, and a duration associated with the one or more electrical stimulation signals.

5. The method of claim 1 further comprising determining, based on one or more of continuous wave Doppler imaging, pulsed-wave imaging, and pulsed-wave Doppler imaging, frequency-modulated continuous wave Doppler imaging, and time-delay spectroscopy imaging, the one or more of ultrasound images and ultrasound signals and the another one or more of ultrasound images and ultrasound signals.

6. The method of claim 1, wherein the activity of the muscle tissue comprises one or more of velocity, strain, strain rate, twitch amplitude, twitch duration, machine-learning decisions determined from signals associated with the muscle tissue, or peak displacement.

7. A method comprising:
determining, based on information associated with one or more of ultrasound images and ultrasound signals, a baseline level of activity of muscle tissue associated with a subject;
stimulating, based on one or more electrical stimulation signals, the muscle tissue, wherein the one or more electrical stimulation signals are associated with a first one or more parameters;
determining, based on information associated with another one or more ultrasound images and ultrasound signals, activity associated with the muscle tissue;
determining, based on the activity associated with the muscle tissue, one or more of a second one or more parameters; and
stimulating, based on another one or more electrical stimulation signals, the muscle tissue, wherein the another one or more electrical stimulation signals are associated with one or more of the second one or more parameters.

8. The method of claim 7, wherein the first one or more parameters comprise a target velocity, a target strain quantity, a target strain rate, a target twitch amplitude, a target twitch duration, and a target peak displacement.

9. The method of claim 7, wherein the second one or more parameters comprise a velocity, a strain quantity, a strain rate, a twitch amplitude, a twitch duration, and a peak displacement associated with the muscle tissue.

10. The method of claim 7 further comprising determining, based on the baseline level of activity and the activity associated with the muscle tissue, an indication of a functional condition associated with the muscle tissue.

11. The method of claim 10, wherein the indication of the functional condition comprises one or more of an indication of fatigue associated with the muscle tissue or recovery associated with the muscle tissue.

12. The method of claim 7 further comprising determining, based on continuous wave Doppler imaging, the one or more of ultrasound images and ultrasound signals and another one or more of ultrasound images and ultrasound signals.

13. The method of claim 7, wherein the activity associated with the muscle tissue comprises one or more of velocity, strain value, strain rate, twitch amplitude, twitch duration, or peak displacement.

14. An apparatus comprising:
one or more processors; and
memory storing processor-executable instructions that, when executed by the one or more processors, cause the apparatus to:
determine, based on information associated with one or more of ultrasound images and ultrasound signals, a baseline level of activity of muscle tissue associated with a subject;
determine, based on information associated with another one or more of ultrasound images and ultrasound signals, activity associated with the muscle tissue; and
determine, based on the baseline level of activity and the activity associated with the muscle tissue, an indication of a functional condition associated with the muscle tissue.

15. The apparatus of claim 14, wherein the indication of the functional condition comprises one or more of an indication of fatigue associated with the muscle tissue or recovery associated with the muscle tissue.

16. The apparatus of claim 14, wherein the processor-executable instructions, when executed by the one or more processors, further cause the apparatus to cause, based on one or more electrical stimulation signals, the activity associated with the muscle tissue.

17. The apparatus of claim 16, wherein the processor-executable instructions that, when executed by the one or more processors, cause the apparatus to cause the activity associated with the muscle tissue further cause the apparatus to send the one or more electrical stimulation signals.

18. The apparatus of claim 16, wherein the processor-executable instructions, when executed by the one or more processors, further cause the apparatus to determine, based on the information associated with the one or more of ultrasound images and ultrasound signals or the information associated with the another one or more of ultrasound images and ultrasound signals, one or more of an amplitude, a frequency, a timing, a metric output my a machine learning model, and a duration associated with the one or more electrical stimulation signals.

19. The apparatus of claim 14, wherein the processor-executable instructions, when executed by the one or more processors, further cause the apparatus to determine, based on continuous wave Doppler imaging, the one or more of ultrasound images and ultrasound signals and the another one or more of ultrasound images and ultrasound signals.

20. The apparatus of claim 14, wherein the processor-executable instructions, when executed by the one or more processors, further cause the apparatus to receive from an ultrasound transducer the one or more of ultrasound images and ultrasound signals and the another one or more of ultrasound images and ultrasound signals.

21. The apparatus of claim 20, wherein the ultrasound transducer is battery powered and comprises dimensions ranging from 0 millimeters to 50 millimeters.

22. The apparatus of claim 16, wherein the activity associated with the muscle tissue comprises one or more of velocity, strain, strain rate, twitch amplitude, twitch duration, or peak displacement.
